(19) United States
(12) Patent Application Publication
(10) Pub. No.: US 2006/0074370 A1
(43) Pub. Date: Apr. 6, 2006

(54) Ocular occluder and method of insertion

(75) Inventor: Stephen Q. Zhou, Irvine, CA (US)

Correspondence Address: FROST BROWN TODD, LLC, 2200 PNC CENTER, 201 E. FIFTH STREET, CINCINNATI, OH, 45202, US

(73) Assignee: Medennium, Inc.

(21) Appl. No.: 10/948,873

(22) Filed: Sep. 24, 2004

(51) Int. Cl. A61M 5/00

(52) U.S. Cl. 604/8

(57) ABSTRACT

An ocular plug design and method of insertion is described for the treatment of dry eye. This ocular plug is generally a narrow rod-like cylinder of appropriate diameter, which may be tapered at one end, for insertion into an ocular channel, such as the punctum or the canaliculus. The plug is prepared from a hydrophilic polymeric material which forms a hydrogel upon absorption of water, but is rigid in its nonhydrated form. The plug is hydrated, formed into a length and diameter which is appropriate for insertion into an ocular channel (i.e., it is elongated), and dried so as to become frozen in its elongated state prior to insertion into the ocular channel. Once inserted into the ocular channel, the plug absorbs water, thereby becoming a hydrogel which is soft and pliable, and it expands to adapt to the size and shape of the patient's punctum or canaliculus. Once the plug expands to the size of a particular ocular channel, the plug is met with resistance from the surrounding tissue. At that point, expansion of the plug ceases and the plug can effectively block tear drainage through the ocular channel.

### TECHNICAL FIELD

The present invention generally relates to a removable intraocular plug used to temporarily close the punctal or canalicular opening of the human eye. The plug can be utilized, for example, in the treatment of keratoconjunctivitis sicca (dry eye). Specifically, the present invention relates to a method for occluding ocular channels by using a plug, made from hydrogel materials, that can adapt to the size and shape of an individual's punctum or canaliculus after insertion.

### BACKGROUND OF THE INVENTION

The human eye includes a complex composition in the form of a tear film. Tears include three basic components: (1) lipids; (2) an aqueous component; and (3) mucin. The absence of any one of these components causes discomfort in the eye and can lead to a temporary or permanent condition known as keratitis sicca (or keratoconjunctivitis sicca), often referred to as dry eye. Dry eye can have a variety of causes but is generally attributed to one or two basic malfunctions. First, the tear ducts leading from the lacrimal glands can be clogged or malfunctioning so that an insufficient amount of tears reaches the eye. For many years, this was generally thought to be the main reason for dry eye. Artificial tears were developed in response to this need. However, the relief to patients using these artificial tears is short-lived and treatment must be readministered several times each hour.

More recently, it has been discovered that, with increasing age, dry eye can also be caused by either the formation of an insufficient or inadequate amount of tears and/or tear components or the inability to maintain effective tear films in the eye. Accordingly, recent therapies have proceeded on the basis that tear production may be inadequate in some individuals and that a significant percentage of dry eye syndrome can be alleviated by slowing down the drainage of the tears through the lacrimal ducts.

Tears are removed from the eye (1) by draining through the upper (2) and lower (3) punctal openings which lead into the canalicular canals (4,5), and ultimately to the lacrimal sac (6) (see FIG. 1). It is there that drainage of tears from the eye can be adjusted. Early attempts at sealing the puncta and/or the canalicular canals involved stitching the puncta shut or using electrical or laser cauterization to seal the puncta and/or the canalicular canals. Although such methodology can provide acceptable drainage results, the procedure is not reversible without reconstructive surgery. Since it is sometimes difficult to determine whether, in a particular patient, the drainage is too great or the tear production is too small, irreversible blockage is not without risk.

One means of temporarily blocking the punctum and canaliculus for the treatment of dry eye is through the use of intracanalicular gelatin implants. “Intracanalicular Gelatin Implants in the Treatment of Keratoconjunctivitis Sicca,” Wallace S. Foulds, Brit. J. Ophthal. (1961) 45:625-7. In this article, Foulds describes how the occlusion of the lacrimal puncta can be performed by the use of and insertion of a fine, water-soluble gelatin rod into the punctal openings. The gelatin rod is formed from a pure powdered gelatin to which a small quantity of distilled water has been added and is heated in a water bath until the gelatin dissolves and a thick gel results. By dipping a cold glass rod into this prepared gelatin, and withdrawing it from the thick gel, fine solid rods of gelatin can be formed. These gelatin rods may be inserted into the canaliculi to provide a temporary blockage. Gelatin rod implants, although very fragile, provide an alternative means for temporarily blocking the canaliculus.

Water-insoluble plugs which can be placed in the punctal openings and into vertical sections of the canalicular canals are disclosed in U.S. Pat. No. 3,949,750, Freeman, issued Apr. 13, 1976. The punctum plug (10) of Freeman is a rod-like plug formed with an oversized lip (11) that dilates and blocks the vertical canaliculus (see FIG. 2). The punctum plug has a relatively large, smooth head portion (12) which functions to prevent the punctum plug from passing into the horizontal portion of the canaliculus. Although these plugs are reversible, they tend to become dislodged quite easily. Further, they are somewhat difficult to insert, and occasionally their size and shape can cause tissue damage during insertion or, if they protrude from the punctum, they can cause irritation to the sclera. The tissue of the punctum can also be damaged by being dilated by the plugs over extended periods of time.

An improvement on the Freeman plugs is disclosed in U.S. Pat. No. 4,959,048, Seder et al., issued Sep. 25, 1990. The Seder et al. patent discloses a preformed plug or channel occluder which is somewhat conical in shape, making it possible to insert the occluder into the opening of the punctum more easily than the devices disclosed by Freeman. Further, the Seder et al. patent discloses that variations in the anatomy of individuals make it desirable to provide a series of occluders having different lengths and/or widths in order to accommodate these differences. Using this approach, ophthalmologists need to measure the actual size of the punctal opening to determine the best size for the punctum plug to be used for each patient and manufacturers must then provide five or more different sizes of punctum plug to meet the ophthalmologist's needs.

Using these prior art plugs, doctors must follow a number of procedures that are not only time consuming but also require a high level of skill. First, doctors need to measure each patient's punctal diameter since this size will vary from patient to patient, and for some patients, there will even be variances in punctum size between the left eye and the right eye (see FIG. 3). This is done by inserting a sizing gauge (13) into the punctum (2). An oversized plug will cause the patient discomfort while an undersized plug will fall out of the patient's eye. Second, doctors need to dilate the punctum (2) and quickly insert the plug, usually within 30 seconds or less (see FIG. 4). The dilation needs to be repeated if the plug fails to be inserted within the 30 seconds and, since the plug is so soft and small, it is often very difficult to complete the insertion within this 30 second time window. FIGS. 4 and 5 show tools (15) which may be used to enlarge the punctum (2) for insertion of the plug. There is clearly a need to simplify this procedure.

In an effort to meet this need, U.S. Pat. No. 6,234,175, Zhou and Wilcox, issued May 22, 2001, discloses a smart ocular plug design and method for insertion which achieves a one-size-fits-all device for blocking the punctum or canaliculus of a patient. This is accomplished by using specifically defined materials having narrowly-defined glass transition temperature and/or melting temperature properties for fabricating the plug.

### SUMMARY OF THE INVENTION

The present invention provides punctal and intracanalicular plugs for occluding punctal openings of an eye to preserve tears for the treatment of dry eye syndrome. The present invention does this using a one-size-fits-all plug design. This eliminates the need for gauging the punctum size in a patent thereby saving time for the doctors. In addition, the one-size-fits-all design requires a much smaller inventory for both manufacturers and service providers.

The present invention also provides a rigid slender plug (20) which is used for insertion into punctal openings (2). By having this configuration, the need for dilating the punctum prior to insertion is eliminated. The insertion of the plug of the present invention is relatively easy. Because the plug of the present invention is a rigid slender rod, it does not need special tools or inserters. Instead, it only requires simple forceps for holding the plug and its insertion into the punctal opening. Finally, the plug of the present invention, when positioned in the punctum or canaliculus, becomes flexible and soft, having a softness and pliability which is similar to that of human eyelid tissue (see FIG. 6). Therefore, it causes minimum or no discomfort to the patient upon insertion.

Specifically, the present invention relates to an ocular occluder for blocking lacrimal flow through the punctum or canaliculus of the human eye, wherein said occluder:
(1) comprises a hydrophilic polymeric material which is capable of forming a hydrogel in water (and preferably, when fully hydrated in water, contains at least about 10% by weight of water);
(2) is capable of being stretched along its length to at least an additional about 50% beyond its initial length;
(3) is rigid at room temperature prior to the absorption of water; and
(4) absorbs water when inserted into the punctum or canaliculus thereby becoming soft and expanding in diameter so as to conform to the shape of said punctum or canaliculus.

This ocular plug of the present invention (20) is a slender rod-like device, generally a cylinder of appropriate diameter, suitable for insertion into the punctum of most patients. It is optionally tapered at the front end for easy insertion into the punctum. It is prepared from a hydrophilic polymeric material which forms a hydrogel upon absorption of water, that has been prestretched and frozen in the stretched form. Once inserted into the ocular channel, the plug absorbs water from its surrounding environment, thereby becoming soft and subsequently starting to recover its initial prestretched shape. The absorption of water by the dry material will cause the plug to increase its dimensions in all directions in proportion to the content of water in the fully hydrated material. This shape deformation, shape recovery and adaptation are illustrated in FIG. 6.

### BRIEF DESCRIPTION OF THE DRAWINGS

FIG. 1 is a representation of the anatomy of a human eye and its associated lacrimal system.

FIG. 2 is a prior art punctum plug used to close the punctal opening to conserve tears in the human eye for treating dry eye symptoms (see U.S. Pat. No. 3,949,750, Freeman, issued Apr. 13, 1976).

FIG. 3 is a gauge used in conjunction with prior art plugs for measuring the diameter of the patient's punctum.

FIG. 4 is a tool used in conjunction with prior art plugs to enlarge the punctum and associated canaliculus prior to insertion of the punctum plug.

FIG. 5 is an inserter tool used in conjunction with prior art plugs for grasping, manipulating and inserting a silicone plug into the punctal opening.

FIG. 6 shows the shape transformation of the elongated needle-like plug of the present invention adapting (as it absorbs water) to its environment in the punctum or canaliculus when a restriction force is present.

FIG. 7 illustrates an embodiment of the present invention wherein a dry hydrophilic polymeric rod (21) is hydrated (22), elongated (23), dried to freeze the rod in the elongated shape (24), cut into pieces of short length (25), and hydrated once again to become a soft hydrogel plug (26) with appropriate dimensions suitable for blocking lacrimal channels.

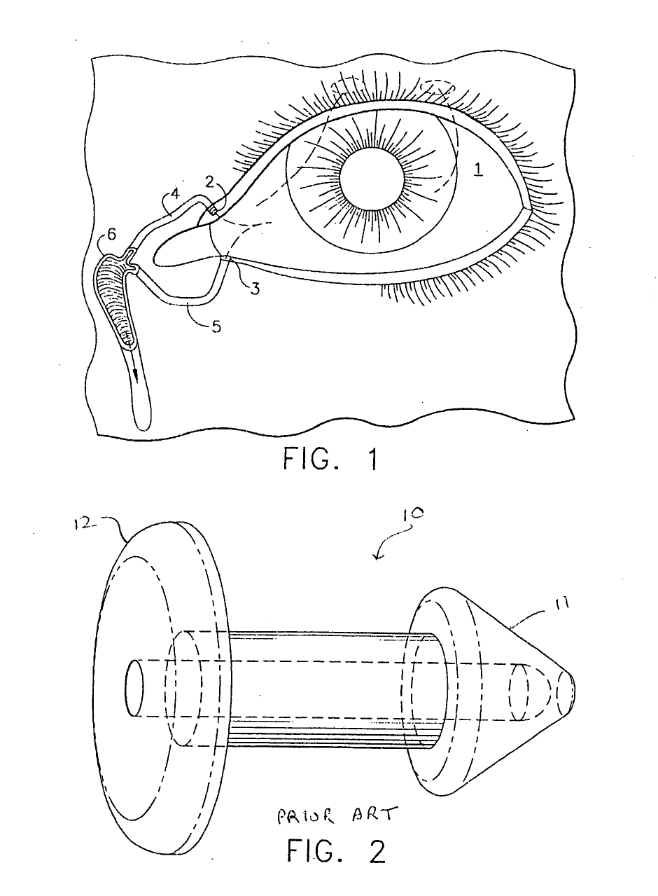

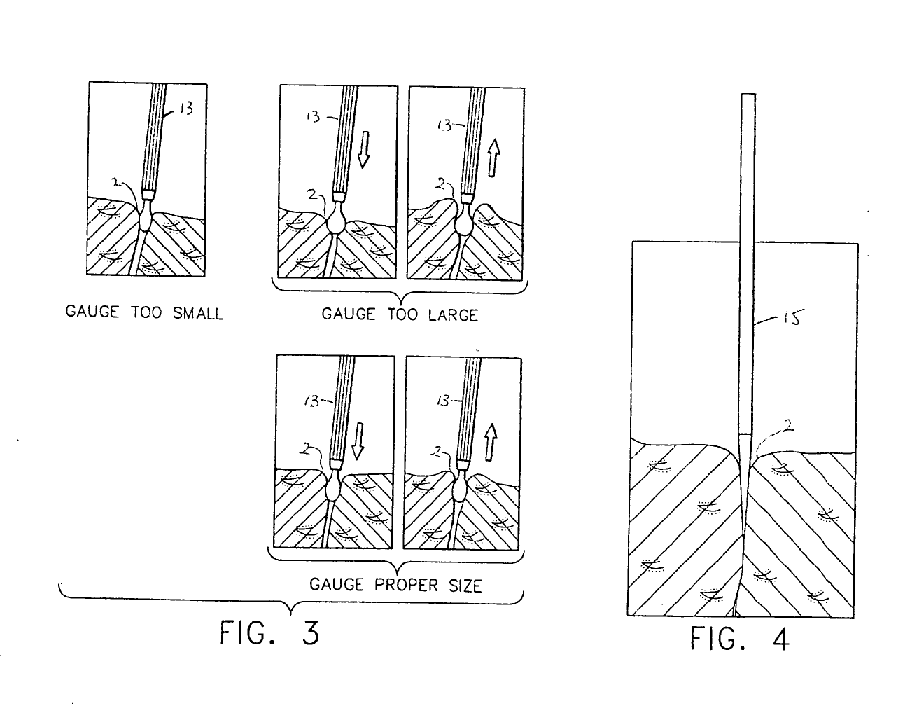

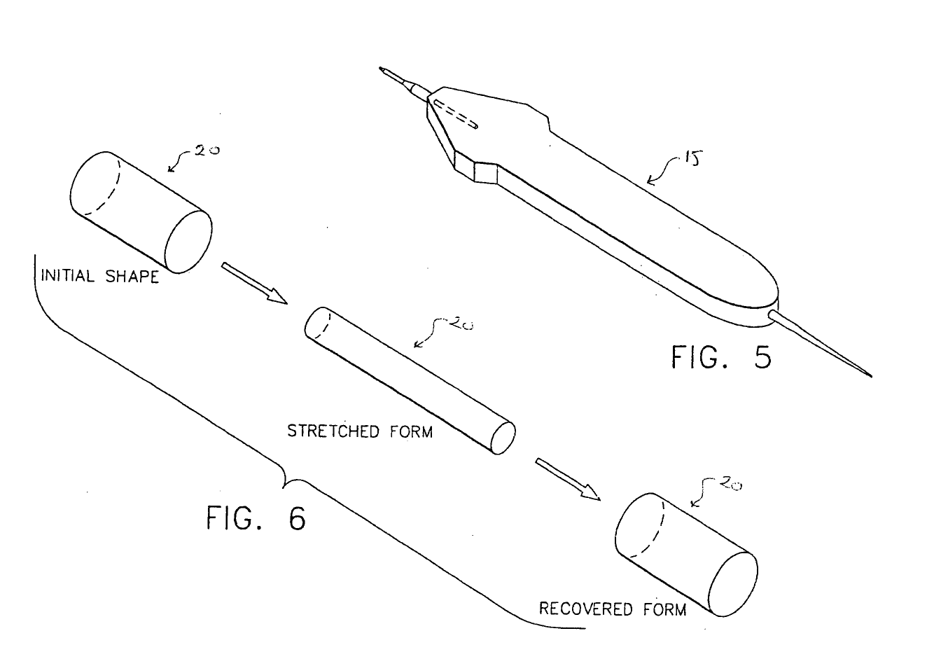

### DETAILED DESCRIPTION OF THE INVENTION

In general terms, the present invention comprises a rod, generally cylindrical, made from a hydrophilic polymeric material which is capable of forming a hydrogel in water, and preferably which is capable of absorbing at least about 10% of its own weight of water to form a hydrogel. Preferred polymers are those which absorb from about 35% to about 60% of their own weight of water, thereby becoming a hydrogel, and which have balanced properties in terms of hardness and elasticity. Generally speaking, hydrogels are defined as water-containing gels characterized by hydrophilicity and insolubility in water. In water, hydrogel-forming polymers swell to an equilibrium volume, while preserving their shape. Examples of such polymeric materials which are suitable for use in the present invention can be found in “Hydrogels in Medicine and Pharmacy,” by Nikolaos A. Peppas, CRC Press, Inc., Boca Raton, Fla., 1986, incorporated herein by reference. Examples of such materials include the polymers and copolymers of: (1) acrylic polymers with hydrophilic substituted pendant groups, such as poly(hydroxyethyl methacrylate), poly(acrylic acid), poly(methacrylic acid); (2) polyethylene backbone polymers having pendant hydrophilic groups, such as poly(vinyl alcohol), poly(N-vinyl-2 pyrrolidone), and poly(vinyl acetate); (3) polymers having hydrophilic groups which are a part of the polymer backbone structure, such as poly(ethylene oxide) and poly(ethylene imine); and (4) polymers having hydrophilic groups being both part of the polymer backbone and also as pendant groups, such as xanthan gum, heparin, hydroxypropylmethyl cellulose, and hyaluronic acid. In addition, these polymers and copolymers can be crosslinked with appropriate crosslinkers to modify their mechanical and physical properties. Particularly preferred materials include the polymers and copolymers of hydroxyethyl methacrylate, hydroxyethyl acrylate, hydroxypropyl methacrylate, hydroxypropyl acrylate, 2,3-dihydroxypropyl methacrylate, methacrylic acid, acrylic acid, N-vinyl-2-pyrrolidone, vinyl alcohol, ethylene oxide, ethylene imine, hydroxypropylmethyl cellulose, methacrylamide, and mixtures of these materials. These hydrogel-forming polymers can be further modified with monomers of less hydrophilicity, such as methyl methacrylate, methyl acrylate, and N,N-dimethyl methacrylamide.

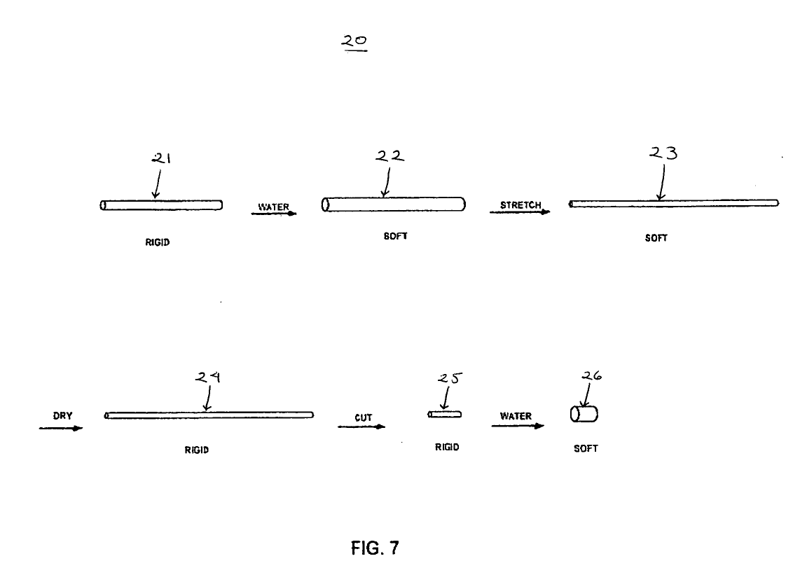

The punctal plugs of the present invention (20) (the terms “plug” and “occluder” are used interchangeably in this application) are generally rods made from the hydrophilic polymeric hydrogel-forming materials described above. These rods tend to be rigid when the polymer is in a nonhydrated form. The initial rigid rod (21) is then hydrated in water so that it become a hydrogel (22). The hydrogel rod is then stretched to at least about an additional 50% (preferably at least about an additional 60%) beyond its initial length (23). For example, if the initial length of the rod is 10 mm, it is stretched to at least 15 mm in length. The hydrogel rod is then dried while in its stretched state so that the rod becomes rigid (24) again, but this time in its stretched form having a smaller diameter than the diameter it had in its initial rigid form. This stretched rigid rod is then cut into appropriate lengths (25) such that when it is fully hydrated the final length will be from about 1 to about 3 mm. This slender rod is used as the plug for insertion into the punctum. Once it is inserted into the punctum, it absorbs water from the surrounding environment and becomes a soft, pliable hydrogel (26). At the same time, the hydrated plug starts to expand in diameter until it adapts to fill the patient's punctum in terms of both its size and shape, thereby plugging the patient's punctum. This result is achieved using the one-size-fits-all design of the present invention. The process of stretching, inserting and utilizing the punctal plugs of the present invention is illustrated in FIG. 7. In this embodiment, the stretching of the initial rod makes the stretched rod have a smaller diameter than that of the initial rod. The hydration of the stretched rod, in the punctum, unlocks the stretching force, causing the rod to return to its initial diameter thereby filling and blocking the punctum of the wearer. The degree of expansion caused by the absorption of water is dependent on the water content of the fully hydrated rod. The higher the water percentage, the more expansion the rod will have. Accordingly, one can design and control the diameter at a desirable level by adjusting either the stretching ability of the initial rod or the water percentage of the fully hydrated rod, or both.

Typically, the occluder of the present invention will be formed in a cylindrical rod shape, although other cross-sectional shapes may be used, as long as the length of the rod is greater than its cross-sectional diameter and, the hydrated rod will expand to fill up the punctum of the wearer. Typically, the occluder will have a diameter of no greater than about 0.7 mm in its stretched form, and preferably will have a diameter of from about 0.3 to about 0.5 mm in its stretched form. Upon absorption of water, the occluder will typically expand to a maximum diameter of from about 1 to about 2 mm, preferably about 1.5 mm. The occluder typically will have a length of from about 3 to about 15 mm in its stretched form and will shrink to a length from about 1 to about 5 mm, preferably from about 1 to about 3 mm when it is fully hydrated.

The occluder may be removed from the eye by injecting a small volume of water or saline solution into the ocular channel using a syringe. This procedure is know to ophthalmologists as the irrigation process.

In order to better understand the teachings of the present invention, the following examples are given for illustration purposes only, and not to limit the scope of the present invention. The dimensions used in the following experiments are not intended to be the most suitable ones for use in human lacrimal channels. Instead, they are used to demonstrate the basic concepts behind the present invention.

### EXAMPLE 1

A mixture of 5 grams 2-hydroxyethyl methacrylate and 10 mg benzoyl peroxide is mixed, degassed and refilled with nitrogen gas three times. The mixture is then transferred to a polypropylene tube having one end thermally pre-sealed. The second end is sealed after the mixture is transferred into the tube. The tube is placed in a preheated oven at a temperature of about 90° C. for 15 hours, then at about 135° C. for 3 hours. The polypropylene tube is cut open and a transparent rigid rod is obtained.

The rod obtained above has a diameter of about 1.2 mm and a length of about 25 mm. The rod is heated on a heating plate until it is soft and elastic. The softened rod is manually stretched by holding both of its ends with forceps. The stretched rod is cooled to room temperature, whereby it became rigid and remains in the stretched form when the forceps were released. The cooling process may be accelerated by dipping the stretched rod into room temperature water. This “freezes” the rod in the stretched form in about 2 seconds.

Both ends of the stretched rod, having been deformed by the forceps during stretching, are removed. Thus, a uniform stretched rod of circular cross-section, with a diameter of about 0.7 mm and a length of about 35 mm is obtained. The stretched rod is cut into five pieces with a length of about 7 mm each. The resulting stretched rod with a diameter of about 0.7 mm and a length of about 7 mm may be used as an occluder for blocking lacrimal flow through the punctum or canaliculus of the human eye.

The occluder prepared above is placed in water at room temperature. When fully hydrated, the occluder has a diameter of about 1.4 mm and a length of about 3 mm. Thus, the rigid stretched rod with a diameter of about 0.7 mm and a length of about 7 mm is expanded by hydration into a soft occluder with a diameter of about 1.4 mm and a length of about 3 mm, i.e., the diameter increased and the length decreased.

During this hydration and expansion process in a patient whose ocular channel is smaller than about 1.4 mm (for example, about 1.2 mm), the expansion of the rod ceases when the resistance force from the surrounding tissue is equal to that of the expansion force of the rod. Therefore, the rod fits snugly with the surrounding tissue to provide long-term occlusion.

The elongation percentages may be measured, for example, using a standard Instron machine.

### EXAMPLES 2-4

Examples 2-4 are prepared in the same manner as described in Example 1 except for the material compositions. Table 1 lists material composition as well as other properties.

TABLE 1

| | Example | | | |
|---|---|---|---|---|
| | 1 | 2 | 3 | 4 |
| Composition* | HEMA | HEMA + | HEMA + | HEMA + 20% |
| | | 0.2% XL | 20% MMA | MMA + 0.2% XL |
| H₂O % | 39% | 36% | 23% | 23% |
| Diameter in | 1.4 | 1.4 | 1.3 | 1.3 |
| mm (wet) | | | | |
| Elongation % | 100% | 45% | 200% | 170% |

*10 mg of benzoyl peroxide was used as an initiator in all compositions
HEMA: hydroxyethyl methacrylate
MA: methyl methacrylate
XL: ethylene glycol dimethacrylate used as a crosslinker
Composition percentage based on weight percentage

### EXAMPLE 5

A composition of 70 wt. % hydroxyethyl methacrylate, 25 wt. % N-benzyl-N-methylacrylate and 5% N,N-dimethylacrylate is polymerized with 90.2 wt. % ethylene glycol dimethacrylate as a crosslinker, and 10 mg benzoyl peroxide as an initiator, under the same conditions as that in Example 1. The resulting material includes 20 wt. % water when fully hydrated and elongation of about 20%.

The compositions of Examples 2-5 may be formed into ocular occluders of the present invention using, for example, the procedure described in Example 1.

What is claimed is:

1. An ocular occluder for blocking lacrimal flow through the punctum or canaliculus of the human eye, wherein said occluder:
(a) comprises a hydrophilic polymeric material which forms a hydrogel, when hydrated in water;
(b) is capable of being stretched along its length to at least an additional about 50% beyond its initial length;
(c) is rigid at room temperature prior to the absorption of water; and
(d) absorbs water when inserted into the punctum or canaliculus thereby becoming soft and expanding in diameter so as to conform to the shape of said punctum or canaliculus.

2. The occluder of claim 1 which contains at least about 10% by weight of water and forms a hydrogel, when fully hydrated in water

3. The occluder of claim 2 which has a cylindrical rod shape.

4. The occluder of claim 3 wherein the rod has a diameter of no greater than about 0.7 mm in its stretched form.

5. The occluder of claim 4 wherein said rod absorbs water to expand to a maximum diameter of about 1.5 mm.

6. The occluder of claim 5 wherein said rod has a length of from about 3 mm to about 15 mm in its stretched form.

7. The occluder of claim 6 wherein said rod has a length of from about 1 mm to about 5 mm when it is fully hydrated.

8. The occluder of claim 2 made from a material selected from polymers and copolymers of acrylic polymers having hydrophilic substituted pendant groups; polyethylene backbone polymers having pendant hydrophilic groups; polymers having hydrophilic groups as part of the polymer backbone structure; and polymers having hydrophilic groups being both part of the polymer backbone and the pendant groups; and mixtures thereof.

9. The occluder according to claim 8 made from a material selected from polymers and copolymers of hydroxyethyl methacrylate, hydroxyethyl acrylate, hydroxypropyl methacrylate, hydroxypropyl acrylate, 2,3-dihydroxypropyl methacrylate, methacrylic acid, acrylic acid, N-vinyl-2-pyrrolidone, vinyl alcohol, ethylene oxide, ethylene imine, hydroxypropylmethyl cellulose, methacrylamide, and mixtures thereof.

10. A method of inserting an occluder into an ocular channel comprising the steps of:
(a) providing an ocular occluder according to claim 2;
(b) hydrating said occluder thereby making it soft and elastic;
(c) stretching said hydrated ocular occluder such that said stretched form has dimensions suitable for insertion into an ocular channel;
(d) drying the stretched ocular occluder until it resolidifies in its stretched form;
(e) inserting said stretched and resolidified rigid occluder into said ocular channel; and
(f) allowing said ocular occluder to absorb water in the ocular channel thereby becoming an elastic hydrogel and conforming to the shape of the ocular channel.

11. The method of claim 10 wherein said ocular occluder has a cylindrical rod shape.

12. The method of claim 11 wherein said rod has a diameter of no greater than about 0.7 mm in its stretched form.

13. The method of claim 12 wherein said rod absorbs water to expand to a maximum diameter of no greater than about 1.5 mm.

14. The method of claim 13 wherein said rod has a length of from about 3 mm to about 15 mm in its stretched form.

15. The method of claim 14 wherein said rod has a length of from about 1 mm to about 5 mm when it is fully hydrated.

16. The method of claim 10 wherein the ocular occluder is made from a material selected from polymers and copolymers of acrylic polymers with hydrophilic substituted pendant groups; polyethylene backbone polymers having pendant hydrophilic groups;
polymers having hydrophilic groups as part of its polymer backbone structure; polymers having hydrophilic groups as part of both its polymer backbone structure and as pendant groups, and mixtures thereof.

17. The method of claim 16 wherein the polymer is selected from polymers and copolymers of hydroxyethyl methacrylate, hydroxyethyl acrylate, hydroxypropyl methacrylate, hydroxypropyl acrylate, 2,3-dihydroxypropyl methacrylate, methacrylic acid, acrylic acid, N-vinyl-2-pyrrolidone, vinyl alcohol, ethylene oxide, ethylene imine, hydroxypropylmethyl cellulose, methacrylamide, and mixtures thereof.

18. A process for manufacturing an ocular occluder for blocking lacrimal flow through the punctum or canaliculus of the human eye comprising the steps of:
(a) providing an ocular occluder according to claim 2 in the form of a cylindrical rod;
(b) hydrating said ocular occluder whereby it becomes soft and elastic;
(c) stretching said hydrated ocular occluder such that said stretched form has dimensions suitable for inserting said ocular occluder into an ocular channel;
(d) allowing the stretched ocular occluder to dry until it resolidifies in its stretched form; and
(e) cutting said stretched rod into an appropriate length for insertion into an ocular channel.

19. The process of claim 18 wherein said rod has a diameter of no greater than about 0.7 mm in its stretched form.

20. The process of claim 19 wherein said rod expands to a diameter of no greater than about 1.5 mm upon absorption of water.

21. The process of claim 20 wherein said rod has a length of from about 3 mm to about 15 mm in its stretched form.

22. The process of claim 21 wherein said rod has a length of from about 1 mm to about 5 mm when it is fully hydrated

23. The process of claim 18 wherein said ocular occluder is made from a polymer selected from polymers and copolymers of acrylic polymers having hydrophilic substituted pendant groups; polyethylene backbone polymers having pendant hydrophilic groups; polymers having hydrophilic groups as part of its polymer backbone structure; polymers having hydrophilic groups as part of both the polymer backbone and the pendant group structure; and mixtures thereof.

24. The process of claim 23 wherein the polymers are selected from polymers and copolymers of hydroxyethyl methacrylate, hydroxyethyl acrylate, hydroxypropyl methacrylate, hydroxypropyl acrylate, 2,3-dihydroxypropyl methacrylate, methacrylic acid, acrylic acid, N-vinyl-2-pyrrolidone, vinyl alcohol, ethylene oxide, ethylene imine, hydroxypropylmethyl cellulose methacrylamide, and mixtures thereof.
